(19) United States
(12) Patent Application Publication
(10) Pub. No.: US 2006/0135482 A1
(43) Pub. Date: Jun. 22, 2006

(54) Use of fosinopril to reduce cardiovascular events in disalysis patients

(76) Inventors: Eric Rougemond, St. Pries (FR); Nadia Amellal, Vincennes (FR)

Correspondence Address: MILLEN, WHITE, ZELANO & BRANIGAN, PC, 2200 CLARENDON BLVD, SUITE 1400, ARLINGTON, VA, 22201, US

(21) Appl. No.: 10/534,052

(22) PCT Filed: Oct. 9, 2003
(86) PCT No.: PCT/EP03/11147

§ 371 Date: May 6, 2005

(30) Foreign Application Priority Data
Nov. 8, 2002 (FR) 0214060

(51) Int. Cl. A61K 31/675

(52) U.S. Cl. 514/91

(57) ABSTRACT

The invention relates to the use of fosinopril or a pharmaceutically acceptable salt thereof, to reduce the risk of occurrence of a cardiovascular event in dialysis patients, in particular blood dialysis patients, especially patients presenting left-ventricular hypertrophy.

The invention relates to a novel therapeutic application of fosinopril to reduce morbidity and mortality of cardiovascular origin in dialysis patients.

Fosinopril, ((2S,4S)-4-cyclohexyl-1-[2-[(R)-[[(S)-2-methyl-1-(propanoyl-oxy)propyl]oxy](4-phenylbutyl)phosphinoyl]acetyl]pyrrolidine-2-carboxylic acid), is a medicament known as an inhibitor of angiotensin converting enzyme (ACE). It is proposed mainly for the treatment of hypertension and also congestive cardiac insufficiency. It is present, as the sole active principle, in a French specialty product known as Fozitec® in France. Fosinopril sodium is also contained in the tablets sold under the name Monopril® in the US.

The efficacy of ACE inhibitors is now well established in a variety of cardiovascular diseases and/or conditions involving a cardiovascular risk.

However, blood dialysis patients suffering from a kidney disease were hitherto excluded from the clinical trials.

Now, it is found that cardiovascular accidents constitute the prime cause of death in dialysis patients and represented 38% of the mortality occurring in this population in 1990 in Canada (Don Mills Ontario, 1992) and 42% in Europe in 1992 (Raine et al., 1992). In this European study, 15% of the deaths were due to a myocardial infarction, 12% to a cardiac insufficiency and 12% to sudden death.

The 1997 USRDS annual data report confirms that cardiovascular accidents still represent almost half of the causes of death in blood dialysis patients, and are mainly due to cardiac arrests (18.2% of the total mortality), myocardial infarctions (9.4%), rhythm disorders (5.8%), cardiomyopathy (4.3%) and coronary atherosclerosis (4.1%). Among the non-cardiac causes of death, strokes represent a major cause of death (6.1%).

The comparatively high death rate by coronary ischaemia due to the existence of a terminal chronic renal insufficiency adds to the differences in susceptibility to coronary diseases observed in the general population (Parfrey et al., 1993; USRDS annual data report, 1997). The risk of cardiovascular disease is even higher in patients whose chronic renal insufficiency is due to diabetes: the death rate caused by coronary insufficiency in diabetic dialysis patients is two or three times higher than that in non-diabetic chronic renal insufficiency patients (Raine et al., 1995 Raine et al., 1992).

Left-ventricular hypertrophy and its consequences, i.e. a reduction in the coronary flow reserve (Rostand et al., 1984), very frequent systolic and/or diastolic congestive cardiac insufficiency (Sica Da et al., 1991), sudden death and other rhythm disorders (Roithinger et al., 1994), explain in large part the comparatively high death rate observed (Ritz et al., 1990). Dilated cardiomyopathy, ischaemic cardiopathy and hyperkinetic hypertrophic cardiomyopathy are other components of uraemic cardiopathy associated with a non-negligible death rate (Parfrey et al., 1996).

Cardiac insufficiency is a frequent complication in terminal renal insufficiency patients undergoing blood dialysis treatment. It involves persistent or recurrent congestive cardiac insufficiency manifestations (dyspnoea, pulmonary oedema and cardiomegaly), whereas the patients are considered as having a “dry” weight.

During the initiation of the dialysis, 31% of the patients show cardiac insufficiency; 56% of these patients have a relapse in the course of an average monitoring of 41 months. The survival of patients with cardiac insufficiency during the introduction of dialysis is 36 months, whereas it is 62 months in the patients who are free of this complication. However, it should be noted that cardiac insufficiency appears in the course of the dialysis treatment in 25% of the patients who did not show any insufficiency at the start of the treatment, within an average onset time of 15 months (Harnett et al., 1995).

The treatment of congestive cardiac insufficiency in blood dialysis patients is not codified. Additional blood dialysis sessions and a reduction of the “dry” weight are frequently involved. Diuretics present no advantage. Digoxin is used empirically without any formal proof of efficacy and with well-known difficulties of practicability.

The inventors are now proposing to combat the occurrence of cardiovascular events with the aid of fosinopril.

Fosinopril differs from other ACE inhibitors on the basis of specific pharmacokinetic properties: it is eliminated in equal parts via the liver and the kidneys if these functions are normal, but its hepatic clearance increases proportionately if the renal function is impaired.

Thus, in patients showing varying degrees of renal insufficiency (including severe insufficiency: creatinine clearance Clcr<10 ml/min), the total clearance of the medicament is reduced (about half of that observed in an individual with normal renal function), but is not correlated with the degree of renal insufficiency (Hui et al., 1991).

Consequently, fosinopril accumulates less than enalapril or lisinopril on repeated administration (10 days) to renal insufficiency patients (Sica Da et al., 1991). This observation is found in cardiac insufficiency patients and renal insufficiency patients (Clcr<30 ml/min), in whom fosinopril administered for 10 days at a rate of 10 mg/day accumulates less than enalapril 2.5 mg/day (AUC_{d10}/AUC_{d1}=1.41 versus 1.96, p=0.02, AUC_{d} meaning the area under the curve), or lisinopril 5 mg/day (1.3 versus 2.57, p<0.001) (Davis et al., 1997).

In dialysis patients (blood dialysis or peritoneal dialysis) with terminal renal insufficiency, the pharmacokinetic parameters of fosinopril remain comparable with those observed in patients with mild, moderate or severe renal insufficiency (Gehr et al., 1991; Gehr et al., 1993).

One subject of the present invention is thus the use of fosinopril or a pharmaceutically acceptable salt thereof to prepare a medicament for reducing the risk of occurrence of a cardiovascular event in a dialysis patient.

The invention also relates to a method for reducing the risk of occurrence of cardiovascular events in a dialysis patient, this method comprising the administration to the said patient of a therapeutically effective amount of fosinopril or of a pharmaceutically acceptable salt thereof.

The “pharmaceutically acceptable salts” that are useful according to the invention are non-toxic salts as described especially in U.S. Pat. No. 4,337,201. These salts especially comprise basic salts, with various organic or mineral bases. Mention may thus be made of the sodium salts, potassium salts, magnesium salts, calcium salts, optionally amine salts (for example salts of dicyclohexylamine, benzathine, N-methyl-D-glucamine or hydrabamine) or salts of amino acids (such as arginine or lysine), and also other salts of the same type (aluminium, iron, bismuth, etc.). Fosinopril sodium is particularly preferred.

The cardiovascular events mentioned above include myocardial infarction, angina (including recent-onset angina, recent rest angina, including decubitus angina and variant angina, and exertional angina), stroke, cardiac insufficiency (that is, especially, cardiac insufficiency decompensation), and also non-fatal or fatal cardiac arrest (including resuscitated sudden death). A death of cardiovascular origin is also considered as such a “cardiovascular event”. The administration of fosinopril or a pharmaceutically acceptable salt thereof moreover makes it possible to reduce the need for angioplasty (coronary or peripheral angioplasty) or bypass (coronary or peripheral bypass).

The treatment according to the invention is more generally directed towards reducing the number and/or severity of cardiovascular events and/or towards increasing the duration of survival without cardiovascular events (or retarding the appearance of the first event) in blood dialysis patients.

The expression “dialysis patients or patients undergoing dialysis” means any human individual or non-human mammal undergoing, in the period of the treatment with fosinopril, dialysis sessions, in particular patients undergoing dialysis on account of a chronic renal insufficiency, in particular in the terminal phase, or on account of an acute renal insufficiency. The renal insufficiency may or may not be of diabetic origin.

The dialyses under consideration may be blood dialyses or peritoneal dialyses.

Dialysis patients, in particular blood dialysis patients, presenting left-ventricular hypertrophy are more particularly targeted.

One group of patients that is particularly targeted also consists of individuals who have been undergoing dialysis treatment, in particular blood dialysis, for at least six months.

The weekly frequency of dialysis sessions in the targeted patients may be at least three.

Dialysis patients presenting a high risk in terms of cardiovascular events are, of course, included. Any man or woman from 50 to 80 years old may thus be treated, in accordance with the invention.

The fosinopril (or a pharmaceutically acceptable salt thereof) may be administered alone or in combination with one or more other medicaments, such as compounds that are useful for the treatment of cardiac insufficiency or for the prevention of cardiovascular disorders (for example erythropoietin, aspirin, statins, etc.) and/or compounds that are useful in the treatment of electrolyte disorders or kidney diseases.

It is generally preferable not to treat the patient in combination with an ACE inhibitor other than fosinopril or a salt thereof.

Fosinopril and/or pharmaceutically acceptable salts thereof are administered in the form of a pharmaceutical composition, in combination with a pharmaceutically acceptable vehicle or excipient.

The terms “excipient” and “pharmaceutically acceptable vehicle” mean any solvent, dispersion medium, absorption retardant, etc. that does not produce a side reaction, for example an allergic reaction, in humans or animals.

The dosage naturally depends on the mode of administration, the formulation chosen and the age and condition of the patient.

If parenteral administration is envisaged, more particularly via injection, the compositions of the invention comprising the active principle(s) are in the form of injectable solutions and suspensions packaged in vials or bottles for slow perfusion. The injection may especially be performed subcutaneously, intramuscularly or intravenously.

For parenteral administration, doses from about 0.005 mg/kg to about 10 mg/kg and preferably from about 0.01 mg/kg to about 1 mg/kg may be envisaged.

For systemic formulations, doses from about 5 to about 2000 mg may be prepared, for administration one to four times a day.

However, the compositions are preferably intended for oral administration.

In this case, the compositions of the invention are in the form of gel capsules, effervescent tablets, plain or coated tablets, sachets, sugar-coated tablets, drinkable vials or solutions, microgranules or sustained-release forms.

The forms for oral administration are prepared by mixing the active substance with various types of excipients or vehicles, such as fillers, crumbling agents (or disintegrating agents), binders, colorants, flavour enhancers and the like, and then formed into the mixture.

The colorant may be any colorant permitted for pharmaceutical use.

Examples of flavour enhancers include cocoa powder, mint, borneol and cinnamon powder.

Examples of binders that may be mentioned are polyvinylpyrrolidone, hydroxypropylmethylcellulose, alginic acid, carbomer, carboxymethylcellulose, dextrin, ethylcellulose, starch, sodium alginate, polymethacrylate, maltodextrin, liquid glucose, magnesium aluminium silicate, hydroxyethylcellulose, hydroxypropylcellulose, ethylcellulose, methylcellulose and guar gum.

It is possible to use alginic acid, sodium carboxymethylcellulose, colloidal silicon dioxide, croscarmellose sodium, crospovidone, guar gum, magnesium aluminium silicate, methylcellulose, microcrystalline cellulose, cellulose powder, pregelatinized starch, sodium alginate or sodium starch glycolate as disintegrating agent.

The fillers are, for example, cellulose, lactose, calcium hydrogen phosphate and microcrystalline cellulose.

The tablets can be obtained in a conventional manner by compressing granules in the presence of one or more lubricants. Suitable lubricants are calcium stearate, glyceryl monostearate, glyceryl palmitostearate, hydrogenated castor oil, hydrogenated plant oil, light mineral oil, magnesium stearate, polyethylene glycol, sodium benzoate, sodium lauryl sulfate, sodium stearyl fumarate, stearic acid, talc and zinc stearate. These tablets can then be coated using polymers in solution or suspension, such as hydroxypropylmethyl-cellulose or ethylcellulose.

The granules used to do this are prepared, for example, by carrying out the wet granulation process using a mixture of the active principles with one or more excipients, such as a binder, a crumbling agent (or disintegrating agent) and a filler.

To obtain hard capsules, the mixture of the active principles with a suitable filler (for example lactose) is incorporated into empty gelatin capsules optionally in the presence of a lubricant, such as magnesium stearate, stearic acid, talc or zinc stearate.

Gel capsules or soft capsules are prepared by dissolving the active principles in a suitable solvent (for example polyethylene glycol), followed by incorporation into soft capsules.

The forms for parenteral administration are obtained in a conventional manner by mixing the active principle(s) with buffers, stabilizers, preserving agents, solubilizers, tonicity agents and suspension agents. In accordance with the known techniques, these mixtures are subsequently sterilized and then packaged in the form of intravenous injections.

As buffer, a person skilled in the art can use buffers based on organophosphate salts.

Examples of suspension agents include methylcellulose, hydroxyethylcellulose, hydroxypropylcellulose, acacia and sodium carboxymethylcellulose.

Examples of solubilizers include castor oil solidified with polyoxyethylene, polysorbate 80, nicotinamide or macrogol.

In addition, stabilizers that are useful according to the invention are sodium sulfite and sodium metasulfite, while mention may be made of sodium p-hydroxybenzoate, sorbic acid, cresol and chlorocresol as preserving agents.

It may be particularly advantageous to formulate the fosinopril or fosinopril sodium with sodium stearyl fumarate or hydrogenated plant oil as lubricant (U.S. Pat. No. 5,006,344) or with stearic acid or zinc stearate, again as lubricant (US 2002/0 131 999).

For the preparation of an oral solution or suspension, the active principles are dissolved or suspended in a suitable vehicle with a dispersant, a humectant, a suspension agent (for example polyvinylpyrrolidone), a preserving agent (such as methylparaben or propylparaben), a flavour enhancer or a colorant.

The tablets may preferably contain from about 5 mg to about 50 mg and preferably from about 5 mg to about 20 mg of fosinopril or a salt thereof.

If the fosinopril or salt thereof is administered orally, a suitable dosage may be defined by the daily administration of about 0.01 mg/kg to about 25 mg/kg of fosinopril or a pharmaceutically acceptable salt thereof. For a person of average weight, a dosage of about 5 to 20 mg per day, preferably 10 mg per day, preferably as a single dosage intake, is particularly advantageous.

One example of a tablet contains 10 mg of fosinopril, as a mixture with lactose, microcrystalline cellulose, povidone, crospovidone and sodium stearyl fumarate.

For the preparation of suppositories, the active principle(s) is (are) mixed in a manner known per se with a suitable base constituent, such as polyethylene glycol or semisynthetic glycerides.

For the preparation of microcapsules, the active principles are combined with suitable diluents, suitable stabilizers, agents for promoting the sustained-release of the active principles or any other type of additive for forming a central core that is then coated with a suitable polymer (for example a water-soluble resin or a water-insoluble resin). The techniques known to those skilled in the art will be used for this purpose.

The microcapsules thus obtained are then optionally formulated in suitable dosage units.

The protocol of a study of the effect of fosinopril on mortality and cardiovascular events in blood dialysis patients presenting left-ventricular hypertrophy is described hereinbelow by way of illustration of the invention.

### OBJECTS AND METHODOLOGY

The main object of the trial was to evaluate the efficacy and tolerance of fosinopril, administered in a daily dosage of from 5 to 20 mg per day for 24 months, on the reduction of the incidence of fatal and non-fatal cardiovascular events in blood dialysis patients presenting left-ventricular hypertrophy (LVH), compared with a placebo, insofar as no treatment to date has proven to be effective in this indication.

The secondary objects were to evaluate the incidence of deaths and hospitalizations, irrespective of the causes, and the duration of event-free survival (retardation of the occurrence of the first event).

This was a controlled, randomized, double-blind phase III multi-centre trial performed in France on two parallel groups of patients, one receiving fosinopril at a daily dosage of 5 to 20 mg/day for 24 months, the other receiving a placebo.

The primary evaluation criterion was a combined criterion comprising the occurrence of at least one of the following events:

death of cardiovascular origin,

non-fatal myocardial infarction,

unstable angina (recent-onset angina, recent rest angina, including decubitus angina and variant angina, and exertional angina),

angioplasty (coronary or peripheral angioplasty),

bypass (coronary or peripheral bypass),

cardiac insufficiency decompensation,

non-fatal cardiac arrest (including resuscitated sudden death),

constituted stroke.

The occurrence of the first event constituted the primary evaluation criterion. However, as far as was possible, the blind nature was maintained and the treatment was continued until the end of the trial, and the patient continued to be monitored according to the modalities envisaged by the protocol.

The secondary evaluation criteria were:

the totality of the events composing the primary evaluation criterion,

the duration of event-free survival (or retardation of appearance of the first event),

the total mortality,

the hospitalizations associated with events, which may or may not be cardiovascular.

### Patients

397 patients were included in the study. The characteristics of these patients are given in Table 1 below:

TABLE 1
patient characteristics

| Characteristic | |
|---|---|
| Age (years) | 66.7 ± 8.04 |
| Women (n/%) | 190 (47.9) |
| Systolic blood pressure (mmHg) | 147.9 ± 21 |
| Diastolic blood pressure (beats/min) | 77.4 ± 11.8 |
| Heart rate (beats/min) | 75.2 ± 12.2 |
| Left-ventricular mass index (g/m²) | 174.1 ± 53.5 |
| History of vascular accidents (n/%) | 27 (6.8) |
| History of coronary artery diseases (n/%) | 52 (13.1) |
| History of peripheral blood vessel diseases (n/%) | 62 (15.6) |
| Patients who smoked during the study (n/%) | 46 (11.6) |
| Diabetes (n/%) | 124 (31.2) |
| Dyslipidaemia (n/%) | 156 (39.3) |
| History of kidney transplant (n/%) | 29 (7.3) |
| Waiting list for kidney transplant (n/%) | 26 (6.6) |
| Treatments (n/%) | |
| Erythropoietin | 313 (78.8) |
| Antidiabetics | 15 (3.8) |
| Insulin | 81 (20.4) |
| Lipid-lowering agents | 100 (25.2) |
| Antihypertensives | 210 (52.9) |
| Size (cm) | 163 ± 9 |
| KT/V | 1.36 ± 0.39 |
| Residual diuresis (ml/day) | 270 ± 375 |
| Predialysis weight (kg) | 71.4 ± 15.6 |
| Inter-dialysis weight gain (kg) | 2.53 ± 1.56 |

The selection criteria were:

men and women between 50 and 80 years old (menopausal women);

patients undergoing blood dialysis treatment for at least six months for terminal renal insufficiency, of diabetic or non-diabetic origin;

weekly frequency of the dialysis sessions at least equal to 3.

The inclusion criteria were:

patients presenting a cardiac hypertrophy defined by a heart mass index of greater than 100 g/m² for the women, and 131 g/m² for the men. The heart mass was determined by means of an echocardiography performed at the latest on the last day of the pre-inclusion period, and at the earliest one month before selection.

The non-inclusion criteria were:

systolic arterial pressure (SAP)≧200 mmHg and/or diastolic arterial pressure (DAP)≧110 mmHg;

permanent increase in transaminases or gamma-GT (>twice the laboratory norm);

clinically significant biological abnormality not directly associated with the terminal renal insufficiency.

### PROTOCOL

On visit V1, the selected patients received for two weeks a placebo under single-blind conditions, at a dose of half a tablet per day. The fosinopril tablet contained 10 mg of fosinopril mixed with lactose, microcrystalline cellulose, povidone, crospovidone and sodium stearyl fumarate (formulation of Fozitec®). The patients satisfying the inclusion criteria were then included in the trial (V2) and were randomized to receive, under double-blind conditions, a test dose of 5 mg of fosinopril or of placebo. The patients who presented after the administration of the test dose a symptomatic hypotension or whose systolic arterial pressure fell below 95 mmHg stopped receiving the trial treatment. All the other patients proceeded into the titration period, which lasted three to six weeks and comprised three to six visits at weekly intervals. These patients were requested to take daily half a tablet (5 mg) of fosinopril or of placebo until the next visit. From V3 to V5, the dosage was increased each week in 5 mg stages, until the maximum dose was reached, which was maintained for the duration of the study. The investigator could delay a dosage stage or reduce (temporarily or definitively) the dosage administered. To do this, three optional additional visits V6, V7 and V8 were envisaged, at the end of which the dosage reached was no longer increased. The subsequent visits V9 to V17 took place in the eighth week and in the third month following inclusion, and then every three months, and were simple evaluation visits, during which the trial treatment dosage could no longer be increased.

During the administration of the test dose, and also at each increase in dosage during the titration period, the fosinopril or placebo was taken in the presence of the investigator, at the earliest two hours before and at the latest immediately before the start of dialysis. The arterial pressure was determined before the start of dialysis. After the administration of the test dose, a measurement of the arterial pressure was performed every 30 minutes for the duration of the dialysis session, and for at least four hours (if necessary up to the sixth hour) after taking the trial product. All the other dosage intakes took place daily in a single dosage intake, preferably in the morning between 8.00 and 10.00, the dialysis days included.

If, at any moment during the trial, the systolic arterial pressure measured in the pre-dialysis period fell below 95 mmHg, or signs of orthostatic hypotension occurred, the investigator interrupted the concomitant hypotensive treatment(s) or reduced the dosage thereof, so as to obtain a systolic arterial pressure of greater than 95 mmHg and to eliminate any manifestation of orthostatic hypotension. In the event of persistence of one or other of these situations, the trial treatment dosage was reduced in 5 mg stages. If, at the dosage of 5 mg/day, the orthostatic hypotension persisted, the trial treatment was stopped.

On visit V1, the demographic data, the disease history and the concomitant treatments were noted, and a full clinical examination and also an ECG were performed.

At the end of the titration period, 300 patients (76%) achieved the target dose of 20 mg.

### BIBLIOGRAPHIC REFERENCES

DAVIS et al., (July 1997) DRUGS, 54(1):103-116

DON MILLS ONTARIO (April 1992) ANNUAL REPORT. HOSPITAL MEDICINE RECORDS INSTITUTE

GEHR et al. (1991) EUR J CLIN PHARMACOL, 41:165-169

GEHR et al. (1993) EUR J CLIN PHARMACOL, 45:431-436

HARNETT et al. (1995) KIDNEY INT, 47:884-890

PARFREY et al. (1993) ACTUALITES NEPHROLOGIQUES, 243-262

PARFREY et al. (1996) NEPHROL DIAL TRANSPLANT, 11 :1277-1285

RAINE et al. (1995) ACTUALITES NEPHROLOGIQUES, 283-305

RAINE et al. (1992) NEPHROL DIAL TRANSPLANT, (SUPP 2):7-35

RITZ et al. (1990) NEPHROL DIAL TRANSPLANT, (SUPP 1):93-97

ROITHINGER et al. (1994) CLIN NEPHROL, 42(5):309-314

ROSTAND et al. (1984) KIDNEY INT, 25:653-659

SICA DA et al. (1991) CLIN PHARMACOKINET, 20(5):420-427

USRDS ANNUAL DATA REPORT (August 1997) AM J KIDNEY DIS, 30 (2) (SUPP 1):S107-S117

1. Use of fosinopril or a pharmaceutically acceptable salt thereof to prepare a medicament for reducing the risk of occurrence of a cardiovascular event in a dialysis patient.

2. Use according to claim 1, in which the cardiovascular event is a cardiac insufficiency.

3. Use according to claim 2, in which the cardiovascular event is a cardiac insufficiency decompensation.

4. Use according to claim 1, in which the cardiovascular event is a myocardial infarction.

5. Use according to claim 1, in which the cardiovascular event is an angina.

6. Use according to claim 1, in which the cardiovascular event is a stroke.

7. Use according to claim 1, in which the cardiovascular event is a non-fatal cardiac arrest.

8. Use according to claim 1, in which the cardiovascular event is a death of cardiovascular origin.

9. Use according to claim 1, in which the patient is a blood dialysis patient.

10. Use according to claim 1, in which the dialysis patient presents left-ventricular hypertrophy.

11. Use according to claim 1, in which the patient undergoes a dialysis treatment on account of a chronic kidney disease in the terminal phase.

12. Use according to claim 1, in which the patient has been undergoing a dialysis treatment for at least six months.

13. Use according to claim 1, in which the patient undergoes a dialysis session at least three times a week.

14. Use according to claim 1, in which the patient is a man or a woman 50 to 80 years old.

15. Use according to claim 1, in which the fosinopril is in the form of fosinopril sodium.

16. Use according to claim 1, in which the medicament is intended for oral administration.

17. Use according to claim 16, in which the medicament comprises from 5 to 20 mg of fosinopril or a pharmaceutically acceptable salt thereof.

18. Use according to claim 16, in which the medicament is intended for a daily administration of about 0.01 mg/kg to about 25 mg/kg of fosinopril or a pharmaceutically acceptable salt thereof.

19. Use according to claim 18, in which the medicament is intended for a daily administration of about 10 mg of fosinopril or a pharmaceutically acceptable salt thereof.
